(19) United States
(12) Patent Application Publication
(10) Pub. No.: US 2023/0177422 A1
(43) Pub. Date: Jun. 8, 2023

(54) BUILDING SYSTEM WITH INFECTIOUS DISEASE HEALTH ANALYSIS BASED ON ATTENDANCE DATA

(71) Applicant: Johnson Controls Tyco IP Holdings LLP, Milwaukee, WI (US)

(72) Inventors: Jonathan D. Douglas, Mequon, WI (US); Kirk H. Drees, Cedarburg, WI (US); Tyler A. Smith, Nolensville, TN (US)

(21) Appl. No.: 17/541,119

(22) Filed: Dec. 2, 2021

(51) Int. Cl. G06Q 10/06; G06Q 10/10; G16H 50/80; G06Q 50/20

(52) U.S. Cl. CPC G06Q 10/06312; G06Q 10/1095; G06Q 50/20; G16H 50/80

(57) ABSTRACT

A building system for a school building, the building system including one or more memory devices storing instructions thereon that, when executed by the one or more processors, cause the one or more processors to receive attendance data indicating whether occupants of the school building are present or absent from the school building, determine, based on the attendance data, an infection risk level of at least one of the school building, a space of the school building, or one or more of the occupants to being infected with an infectious disease present in a population, and perform one or more operations for causing the infection risk level to be reduced in the school building.

### BACKGROUND

The present disclosure relates generally to building systems for a school building. The present disclosure relates more particularly to infectious disease health analysis for a school building. Emergency situations, such as a pandemic, where an infectious disease is spreading, can create stress on organizations. A pandemic can disrupt the activities of occupants within office buildings, schools, apartments, or other buildings where occupants live, work, learn, or otherwise congregate. Furthermore, some diseases, even after they are no longer present in a society, may have lasting impacts on how occupants interact within a building and/or facility. Accordingly, buildings and/or facilities must adapt to operate and implement policies to respond to an infectious diseases.

### SUMMARY

One implementation of the present disclosure is a building system for a school building, the building system including one or more memory devices storing instructions thereon that, when executed by the one or more processors, cause the one or more processors to receive attendance data indicating whether occupants of the school building are present or absent from the school building. The instructions cause the one or more processors to determine, based on the attendance data, an infection risk level of at least one of the school building, a space of the school building, or one or more of the occupants to being infected with an infectious disease present in a population and perform one or more operations for causing the infection risk level to be reduced in the school building.

In some embodiments, the attendance data includes identities of students and class locations of classes that the students are present at or absent from.

In some embodiments, the instructions cause the one or more processors to receive class schedules of classes of the school building, the class schedules indicating the classes, identities of students associated to the classes, room assignments of the classes, a class length of the classes, and teachers of the classes and determine, based on the attendance data and the class schedules, the infection risk level of the at least one of the space or the occupants.

In some embodiments, the occupants include students, and wherein the instructions cause the one or more processors to determine a space infection level for the space of the school building based on the attendance data and a school schedule indicating events occurring in certain spaces of the school building at certain times with certain students of the students and implement, responsive to determining the space infection level of the space is above a predefined amount, at least one of scheduling cleaning for the space, implementing updated environmental control of the space, or recommending additional filtration equipment for the space.

In some embodiments, the instructions cause the one or more processors to determine, based on the attendance data, the infection risk level of groups of students of the school building and recommend one or more first groups of students of the groups of students attend in person class and one or more second groups of students of the groups of students attend remote class based on the infection risk level of the groups of students.

In some embodiments, the infectious disease is a human transmitted disease transmitted between the occupants via air, physical contact with surfaces, and/or physical contact between the occupants.

In some embodiments, the one or more operations include operating one or more pieces of equipment in the school building to reduce the infection risk level.

In some embodiments, the one or more operations include generating a recommendation for adding or retrofitting equipment of the school building, wherein the added or retrofit equipment operate to reduce the infection risk level.

In some embodiments, the one or more operations include scheduling cleaning in one or more areas of the school building.

In some embodiments, the instructions cause the one or more processors to determine one or more recommendations for one or more second school buildings based on the infection risk level of the school building.

In some embodiments, the instructions cause the one or more processors to determine the infection risk level for a first occupant based on an amount of time the first occupant spent in a same space as a second occupant, the amount of time determined using the attendance data.

In some embodiments, the one or more operations include either deploying or recommending deployment of a portable filtration device.

In some embodiments, the occupants include students, and wherein the instructions cause the one or more processors to perform contact tracing to identify one or more potentially impacted students of the students by identifying one or more absent students based on the attendance data and identifying the one or more potentially impacted students based on the one or more absent students and a school schedule, wherein the school schedule indicates interaction events between the students and indicates that the one or more potentially impacted students had direct or indirect interaction with the one or more absent students.

In some embodiments, the school schedule indicates that the one or more potentially impacted students were in a same space of the school building as the one or more absent students.

In some embodiments, the instructions cause the one or more processors to determine the infection risk level further based on a physical design and/or construction of the school building and equipment operating settings of equipment of the school building.

In some embodiments, the instructions cause the one or more processors to determine the infection risk level further based on third party health data of a geographic region where the school building is located.

In some embodiments, the occupants include students, and wherein the instructions cause the one or more processors to determine one or more potentially infected students of the students based on the attendance data, determine groups of students that ride school busses, identify one or more groups of the groups of students that include the one or more potentially infected students, and determine the infection risk level based on the one or more groups of the groups of students that include the one or more potentially infected students.

In some embodiments, the instructions cause the one or more processors to identify one or more school buses of the school busses that the one or more potentially infected students have ridden on or are riding on and perform at least one of environmental control of the one or more school buses or scheduling cleaning for the one or more school buses.

In some embodiments, the one or more operations include determining an infectious disease prevention policy recommendation for the school building using the infection risk level.

In some embodiments, the infectious disease prevention policy recommendation is at least one of a mask requirement, a remote school policy, an in person policy, or a social distancing requirement.

In some embodiments, the occupants include students and staff members, the staff members including teachers, and wherein the instructions cause the one or more processors to perform contact tracing to identify one or more potentially impacted students of the students by identifying an absent staff member based on the attendance data and identifying the one or more potentially impacted students based on the absent staff member and a school schedule, wherein the school schedule indicates interaction events between the one or more potentially impacted students and the absent staff member and indicates that the one or more potentially impacted students had direct or indirect interaction with the absent staff member.

In some embodiments, the instructions cause the one or more processors to identify the one or more potentially impacted students using the attendance data and the school schedule, and wherein the school schedule includes data indicating classes that the one or more potentially impacted students attended and data indicating classes that the absent staff member taught.

Another implementation of the present disclosure is a method including receiving, by a processing circuit, attendance data indicating whether occupants of a school building are present or absent from the school building, determining, by the processing circuit, based on the attendance data, an infection risk level of at least one of the school building, a space of the school building, or one or more of the occupants to being infected with an infectious disease present in a population, and performing, by the processing circuit, one or more operations for causing the infection risk level to be reduced in the school building.

In some embodiments, the attendance data includes identities of students and class locations of classes that the students are present at or absent from.

In some embodiments, the method further includes receiving, by the processing circuit, class schedules of classes of the school building, the class schedules indicating the classes, identities of students associated to the classes, room assignments of the classes, a class length of the classes, and teachers of the classes and determining, by the processing circuit, based on the attendance data and the class schedules, the infection risk level of the at least one of the space or the occupants.

In some embodiments, the occupants include students. In some embodiments, the method includes determining, by the processing circuit, a space infection level for the space of the school building based on the attendance data and a school schedule indicating events occurring in certain spaces of the school building at certain times with certain students of the students and implementing, by the processing circuit, responsive to determining the space infection level of the space is above a predefined amount, at least one of scheduling cleaning for the space, implementing updated environmental control of the space, or recommending additional filtration equipment for the space.

In some embodiments, the method includes determining, by the processing circuit, based on the attendance data, the infection risk level of groups of students of the school building and recommending, by the processing circuit, one or more first groups of students of the groups of students attend in person class and one or more second groups of students of the groups of students attend remote class based on the infection risk level of the groups of students.

In some embodiments, the one or more operations include operating one or more pieces of equipment in the school building to reduce the infection risk level.

In some embodiments, the one or more operations include determining an infectious disease prevention policy recommendation for the school building using the infection risk level.

Another implementation of the present disclosure is a building system for a building, the building system including one or more memory devices storing instructions thereon that, when executed by the one or more processors, cause the one or more processors to receive attendance data indicating whether occupants of the building are present or absent from the building, determine, based on the attendance data, an infection risk level of at least one of the building, a space of the building, or one or more of the occupants to being infected with an infectious disease present in a population, and perform one or more operations for causing the infection risk level to be reduced in the building.

### BRIEF DESCRIPTION OF THE DRAWINGS

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1 is a drawing of a building equipped with a HVAC system, according to an exemplary embodiment.

FIG. 2 is a block diagram of a building automation system (BAS) that may be used to monitor and/or control the building of FIG. 1, according to an exemplary embodiment.

FIG. 3 is a schematic drawing of a floor of a school building, according to an exemplary embodiment.

FIG. 4 is a block diagram of a school health analysis system that can determine disease risk scores for the school building of FIG. 3 based on attendance data of the school building, according to an exemplary embodiment.

FIG. 5 is a flow diagram of a process of determining an infection risk level based on attendance data for the school building of FIG. 3, according to an exemplary embodiment.

FIG. 6 is a flow diagram of a process of using an infection risk level to determine and set a disease prevention policy for the school building of FIG. 3, according to an exemplary embodiment.

FIG. 7 is a flow diagram of a process of performing contact tracing for the school building of FIG. 3 based on attendance data for the school building and a schedule of the school building, according to an exemplary embodiment.

### DETAILED DESCRIPTION

### Overview

Referring generally to the FIGURES, a school building system of a school with infectious disease health analysis is shown, according to an exemplary embodiment. In some embodiments, the school may need to set policies for managing an infectious disease in the school. For example, when an infectious disease is present in a population, e.g., coronavirus (COVID-19), diphtheria, Ebola, influenza, measles, an airborne disease, a human contact transmitted disease, a human to surface contact transmitted disease, etc. this may have negative effects on the school. The disease may spread via the air, through physical contact with surfaces, and/or physical contact between occupants. For example, individuals making decisions for the school may need to make decisions regarding whether school should be in or out of session, whether classes should be remote or in person, whether social distancing is needed at the school, whether masks are needed at the school, etc.

The school building system can be configured to analyze risk levels associated with the school building in relation to occupants of the school (e.g., teachers and/or students) contracting and/or spreading the infectious disease. The school building system can determine infectious disease risk levels for the school building, spaces of the school building, teachers of the school, students of the school, etc. and set the policies for managing the infectious disease in the school building based on the infectious disease risk level.

In some embodiments, the school building system can determine the infectious disease risk levels based on attendance data of the school. The school building system can determine, based on the attendance data, which and how many students are absent from the school (e.g., on a particular day, during a particular week, during a particular month, etc.). If a student is absent from a school and/or class, this may indicate that the student has contracted the infectious disease and is sick. Based on the number of absent students, their class schedules, and/or their identifies, the school building system can determine infectious disease risk levels for the school, for specific spaces of the school, or specific occupants of the school (e.g., specific students or teachers of the school).

In some embodiments, the infectious disease risk levels are further based on information such as a school schedule of a building, local health department data for a geographic region that the school is located (e.g., level of population infected), etc. This information can, in some embodiments, be used with, or in place of, the attendance data.

In addition to setting school policies for the school, the building school system can be configured to set cleaning schedules and/or cleaning levels for various buildings and/or building spaces of a school. For example, the building school system can determine infectious disease risk levels for various spaces of a building, e.g., based on how many potentially infected students (e.g., absent students) were in the various spaces previously. If the infectious disease risk level is over a predefined amount, the building school system can schedule cleaning for the spaces. In some embodiments, the level of the infectious disease risk level can set a level of cleaning needed for the space, e.g., whether the space needs fumigation, cleaning by hand, a quick cleaning, a long deep clean, etc.

In some embodiments, the school building system can set a school policy based on the infectious disease risk level. The building school system can recommend policies such as whether school should be in or out of session, whether classes should be remote or in person, whether social distancing is needed, whether masks are needed, etc. The policy recommendation can be provided to a user device of a principle and/or administrator to be approved, modified, or rejected. The building school system can communicate the policy to user devices of students, teachers, staff, etc. once the policy is approved.

In some embodiments, the school building system can be configured to compare the disease related performance of one school against another school. For example, an infectious disease risk level for one school can be compared to the levels for a group of schools to determine whether the one school is performing well or not. In some embodiments, the school building system can be configured to recommend the control settings and/or policy settings of a high performing school (e.g., a school with a lowest risk score) for a group of schools to other schools.

In some embodiments, the school building system can be implemented for a factory, office building, or other workplace with groups of employees. A system can implement the techniques described herein for a workplace and analyze attendance information (e.g., employees taking sick days, whether employees have punched in or not, etc.) for the workplace to determine infectious disease risk scores for workplace, spaces of the workplace, and/or employees. The scores can be used by the system to set policies for the workplace, implement control settings for the workplace, etc. In some embodiments, the scores can be determined based on locations of employees in the workplace, e.g., office location, workstation location, factory floor location, etc. This location information can be used by the system to determine the scores.

### Building Management System and HVAC System

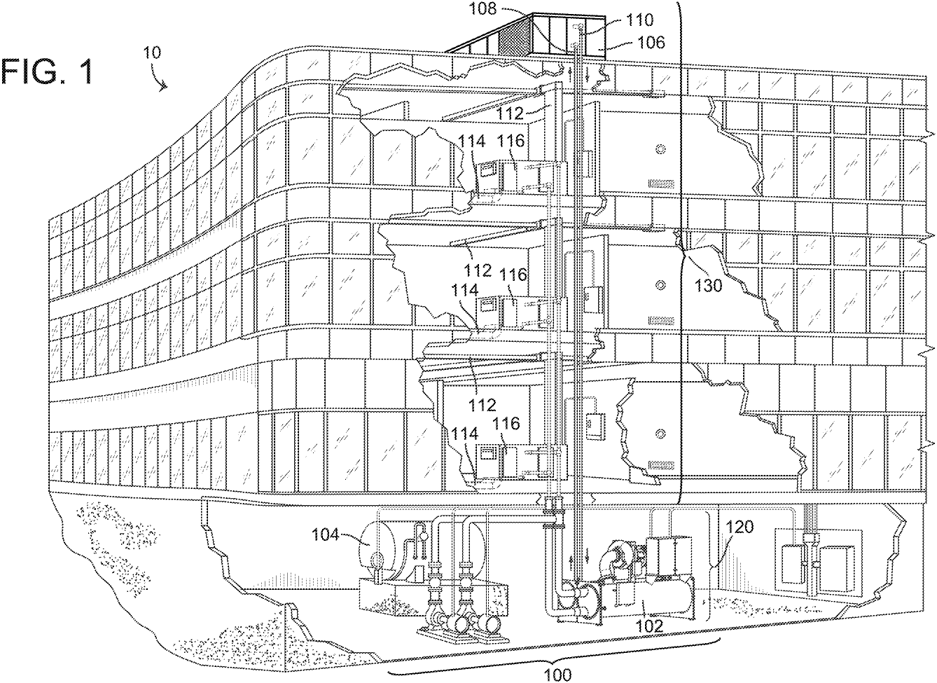

Referring now to FIG. 1, an exemplary building management system (BMS) and HVAC system in which the systems and methods of the present invention can be implemented are shown, according to an exemplary embodiment. Referring particularly to FIG. 1, a perspective view of a building 10 is shown. Building 10 is served by a BMS. A BMS is, in general, a system of devices configured to control, monitor, and manage equipment in or around a building or building area. A BMS can include, for example, a HVAC system, a security system, a lighting system, a fire alerting system, and/or any other system that is capable of managing building functions or devices, or any combination thereof.

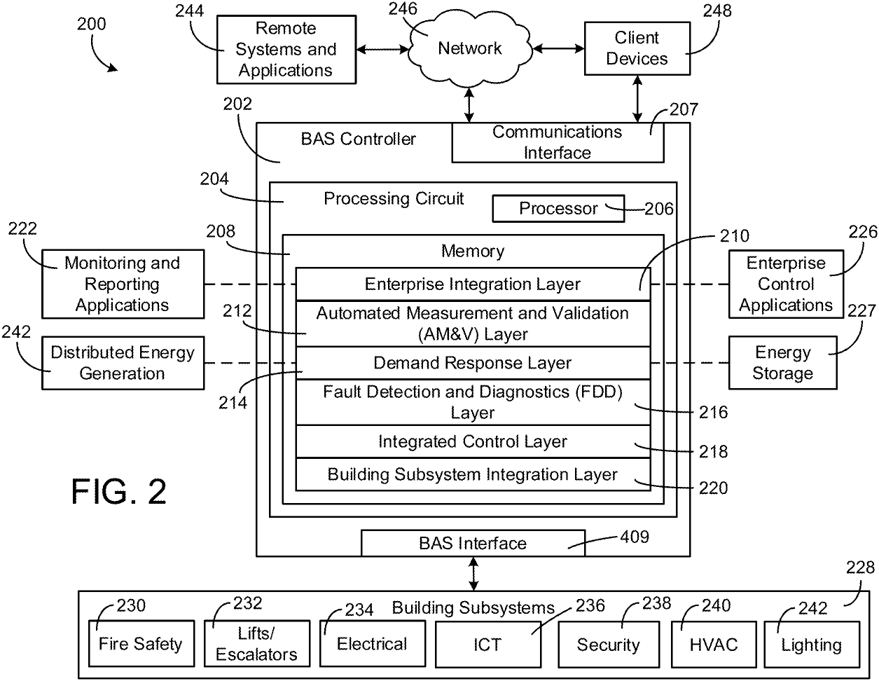

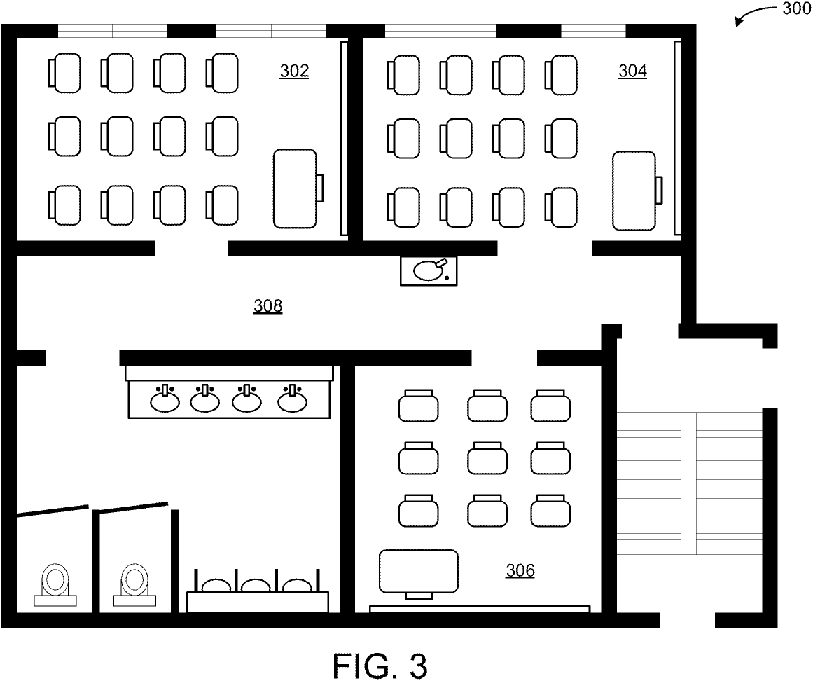

The BMS that serves building 10 includes an HVAC system 100. HVAC system 100 can include HVAC devices (e.g., heaters, chillers, air handling units, pumps, fans, thermal energy storage, etc.) configured to provide heating, cooling, ventilation, or other services for building 10. For example, HVAC system 100 is shown to include a waterside system 120 and an airside system 130. Waterside system 120 can provide a heated or chilled fluid to an air handling unit of airside system 130. Airside system 130 can use the heated or chilled fluid to heat or cool an airflow provided to building 10. An exemplary waterside system and airside system which can be used in HVAC system 100 are described in greater detail with reference to FIGS. 2-3.

HVAC system 100 is shown to include a chiller 102, a boiler 104, and a rooftop air handling unit (AHU) 106. Waterside system 120 can use boiler 104 and chiller 102 to heat or cool a working fluid (e.g., water, glycol, etc.) and can circulate the working fluid to AHU 106. In various embodiments, the HVAC devices of waterside system 120 can be located in or around building 10 (as shown in FIG. 1) or at an offsite location such as a central plant (e.g., a chiller plant, a steam plant, a heat plant, etc.). The working fluid can be heated in boiler 104 or cooled in chiller 102, depending on whether heating or cooling is required in building 10. Boiler 104 can add heat to the circulated fluid, for example, by burning a combustible material (e.g., natural gas) or using an electric heating element. Chiller 102 can place the circulated fluid in a heat exchange relationship with another fluid (e.g., a refrigerant) in a heat exchanger (e.g., an evaporator) to absorb heat from the circulated fluid. The working fluid from chiller 102 and/or boiler 104 can be transported to AHU 106 via piping 108.

AHU 106 can place the working fluid in a heat exchange relationship with an airflow passing through AHU 106 (e.g., via one or more stages of cooling coils and/or heating coils). The airflow can be, for example, outside air, return air from within building 10, or a combination of both. AHU 106 can transfer heat between the airflow and the working fluid to provide heating or cooling for the airflow. For example, AHU 106 can include one or more fans or blowers configured to pass the airflow over or through a heat exchanger containing the working fluid. The working fluid can then return to chiller 102 or boiler 104 via piping 110.

Airside system 130 can deliver the airflow supplied by AHU 106 (i.e., the supply airflow) to building 10 via air supply ducts 112 and can provide return air from building 10 to AHU 106 via air return ducts 114. In some embodiments, airside system 130 includes multiple variable air volume (VAV) units 116. For example, airside system 130 is shown to include a separate VAV unit 116 on each floor or zone of building 10. VAV units 116 can include dampers or other flow control elements that can be operated to control an amount of the supply airflow provided to individual zones of building 10. In other embodiments, airside system 130 delivers the supply airflow into one or more zones of building 10 (e.g., via supply ducts 112) without using intermediate VAV units 116 or other flow control elements. AHU 106 can include various sensors (e.g., temperature sensors, pressure sensors, etc.) configured to measure attributes of the supply airflow. AHU 106 can receive input from sensors located within AHU 106 and/or within the building zone and can adjust the flow rate, temperature, or other attributes of the supply airflow through AHU 106 to achieve setpoint conditions for the building zone.

Referring now to FIG. 2, a block diagram of a building automation system (BAS) 200 is shown, according to an exemplary embodiment. BAS 200 can be implemented in building 10 to automatically monitor and control various building functions. BAS 200 is shown to include BAS controller 202 and building subsystems 228. Building subsystems 228 are shown to include a building electrical subsystem 234, an information communication technology (ICT) subsystem 236, a security subsystem 238, a HVAC subsystem 240, a lighting subsystem 242, a lift/escalators subsystem 232, and a fire safety subsystem 230. In various embodiments, building subsystems 228 can include fewer, additional, or alternative subsystems. For example, building subsystems 228 can also or alternatively include a refrigeration subsystem, an advertising or signage subsystem, a cooking subsystem, a vending subsystem, a printer or copy service subsystem, or any other type of building subsystem that uses controllable equipment and/or sensors to monitor or control building 10. In some embodiments, building subsystems 228 include a waterside system and/or an airside system. A waterside system and an airside system are described with further reference to U.S. Pat. Application No. 15/631,830 filed Jun. 23, 2017, the entirety of which is incorporated by reference herein.

Each of building subsystems 228 can include any number of devices, controllers, and connections for completing its individual functions and control activities. HVAC subsystem 240 can include many of the same components as HVAC system 100, as described with reference to FIG. 1. For example, HVAC subsystem 240 can include a chiller, a boiler, any number of air handling units, economizers, field controllers, supervisory controllers, actuators, temperature sensors, and other devices for controlling the temperature, humidity, airflow, or other variable conditions within building 10. Lighting subsystem 242 can include any number of light fixtures, ballasts, lighting sensors, dimmers, or other devices configured to controllably adjust the amount of light provided to a building space. Security subsystem 238 can include occupancy sensors, video surveillance cameras, digital video recorders, video processing servers, intrusion detection devices, access control devices and servers, or other security-related devices.

Still referring to FIG. 2, BAS controller 202 is shown to include a communications interface 207 and a BAS interface 209. Interface 207 can facilitate communications between BAS controller 202 and external applications (e.g., monitoring and reporting applications 222, enterprise control applications 226, remote systems and applications 244, applications residing on client devices 248, etc.) for allowing user control, monitoring, and adjustment to BAS controller 202 and/or subsystems 228. Interface 207 can also facilitate communications between BAS controller 202 and client devices 248. BAS interface 209 can facilitate communications between BAS controller 202 and building subsystems 228 (e.g., HVAC, lighting security, lifts, power distribution, business, etc.).

Interfaces 207, 209 can be or include wired or wireless communications interfaces (e.g., jacks, antennas, transmitters, receivers, transceivers, wire terminals, etc.) for conducting data communications with building subsystems 228 or other external systems or devices. In various embodiments, communications via interfaces 207, 209 can be direct (e.g., local wired or wireless communications) or via a communications network 246 (e.g., a WAN, the Internet, a cellular network, etc.). For example, interfaces 207, 209 can include an Ethernet card and port for sending and receiving data via an Ethernet-based communications link or network. In another example, interfaces 207, 209 can include a Wi-Fi transceiver for communicating via a wireless communications network. In another example, one or both of interfaces 207, 209 can include cellular or mobile phone communications transceivers. In one embodiment, communications interface 207 is a power line communications interface and BAS interface 209 is an Ethernet interface. In other embodiments, both communications interface 207 and BAS interface 209 are Ethernet interfaces or are the same Ethernet interface.

Still referring to FIG. 2, BAS controller 202 is shown to include a processing circuit 204 including a processor 206 and memory 208. Processing circuit 204 can be communicably connected to BAS interface 209 and/or communications interface 207 such that processing circuit 204 and the various components thereof can send and receive data via interfaces 207, 209. Processor 206 can be implemented as a general purpose processor, an application specific integrated circuit (ASIC), one or more field programmable gate arrays (FPGAs), a group of processing components, or other suitable electronic processing components.

Memory 208 (e.g., memory, memory unit, storage device, etc.) can include one or more devices (e.g., RAM, ROM, Flash memory, hard disk storage, etc.) for storing data and/or computer code for completing or facilitating the various processes, layers and modules described in the present application. Memory 208 can be or include volatile memory or non-volatile memory. Memory 208 can include database components, object code components, script components, or any other type of information structure for supporting the various activities and information structures described in the present application. According to an exemplary embodiment, memory 208 is communicably connected to processor 206 via processing circuit 204 and includes computer code for executing (e.g., by processing circuit 204 and/or processor 206) one or more processes described herein.

In some embodiments, BAS controller 202 is implemented within a single computer (e.g., one server, one housing, etc.). In various other embodiments BAS controller 202 can be distributed across multiple servers or computers (e.g., that can exist in distributed locations). Further, while FIG. 2 shows applications 222 and 226 as existing outside of BAS controller 202, in some embodiments, applications 222 and 226 can be hosted within BAS controller 202 (e.g., within memory 208).

Still referring to FIG. 2, memory 208 is shown to include an enterprise integration layer 210, an automated measurement and validation (AM&V) layer 212, a demand response (DR) layer 214, a fault detection and diagnostics (FDD) layer 216, an integrated control layer 218, and a building subsystem integration later 220. Layers 210-220 is configured to receive inputs from building subsystems 228 and other data sources, determine optimal control actions for building subsystems 228 based on the inputs, generate control signals based on the optimal control actions, and provide the generated control signals to building subsystems 228 in some embodiments. The following paragraphs describe some of the general functions performed by each of layers 210-220 in BAS 200.

Enterprise integration layer 210 can be configured to serve clients or local applications with information and services to support a variety of enterprise-level applications. For example, enterprise control applications 226 can be configured to provide subsystem-spanning control to a graphical user interface (GUI) or to any number of enterprise-level business applications (e.g., accounting systems, user identification systems, etc.). Enterprise control applications 226 can also or alternatively be configured to provide configuration GUIs for configuring BAS controller 202. In yet other embodiments, enterprise control applications 226 can work with layers 210-220 to optimize building performance (e.g., efficiency, energy use, comfort, or safety) based on inputs received at interface 207 and/or BAS interface 209.

Building subsystem integration layer 220 can be configured to manage communications between BAS controller 202 and building subsystems 228. For example, building subsystem integration layer 220 can receive sensor data and input signals from building subsystems 228 and provide output data and control signals to building subsystems 228. Building subsystem integration layer 220 can also be configured to manage communications between building subsystems 228. Building subsystem integration layer 220 translate communications (e.g., sensor data, input signals, output signals, etc.) across multi-vendor/multi-protocol systems.

Demand response layer 214 can be configured to optimize resource usage (e.g., electricity use, natural gas use, water use, etc.) and/or the monetary cost of such resource usage in response to satisfy the demand of building 10. The optimization can be based on time-of-use prices, curtailment signals, energy availability, or other data received from utility providers, distributed energy generation systems 224, from energy storage 227, or from other sources. Demand response layer 214 can receive inputs from other layers of BAS controller 202 (e.g., building subsystem integration layer 220, integrated control layer 218, etc.). The inputs received from other layers can include environmental or sensor inputs such as temperature, carbon dioxide levels, relative humidity levels, air quality sensor outputs, occupancy sensor outputs, room schedules, and the like. The inputs can also include inputs such as electrical use (e.g., expressed in kWh), thermal load measurements, pricing information, projected pricing, smoothed pricing, curtailment signals from utilities, and the like.

According to an exemplary embodiment, demand response layer 214 includes control logic for responding to the data and signals it receives. These responses can include communicating with the control algorithms in integrated control layer 218, changing control strategies, changing setpoints, or activating/deactivating building equipment or subsystems in a controlled manner. Demand response layer 214 can also include control logic configured to determine when to utilize stored energy. For example, demand response layer 214 can determine to begin using energy from energy storage 227 just prior to the beginning of a peak use hour.

In some embodiments, demand response layer 214 includes a control module configured to actively initiate control actions (e.g., automatically changing setpoints) which minimize energy costs based on one or more inputs representative of or based on demand (e.g., price, a curtailment signal, a demand level, etc.). In some embodiments, demand response layer 214 uses equipment models to determine an optimal set of control actions. The equipment models can include, for example, thermodynamic models describing the inputs, outputs, and/or functions performed by various sets of building equipment. Equipment models can represent collections of building equipment (e.g., subplants, chiller arrays, etc.) or individual devices (e.g., individual chillers, heaters, pumps, etc.).

Demand response layer 214 can further include or draw upon one or more demand response policy definitions (e.g., databases, XML files, etc.). The policy definitions can be edited or adjusted by a user (e.g., via a graphical user interface) so that the control actions initiated in response to demand inputs can be tailored for the user’s application, desired comfort level, particular building equipment, or based on other concerns. For example, the demand response policy definitions can specify which equipment can be turned on or off in response to particular demand inputs, how long a system or piece of equipment should be turned off, what setpoints can be changed, what the allowable setpoint adjustment range is, how long to hold a high demand setpoint before returning to a normally scheduled setpoint, how close to approach capacity limits, which equipment modes to utilize, the energy transfer rates (e.g., the maximum rate, an alarm rate, other rate boundary information, etc.) into and out of energy storage devices (e.g., thermal storage tanks, battery banks, etc.), and when to dispatch on-site generation of energy (e.g., via fuel cells, a motor generator set, etc.).

Integrated control layer 218 can be configured to use the data input or output of building subsystem integration layer 220 and/or demand response later 214 to make control decisions. Due to the subsystem integration provided by building subsystem integration layer 220, integrated control layer 218 can integrate control activities of the subsystems 228 such that the subsystems 228 behave as a single integrated supersystem. In an exemplary embodiment, integrated control layer 218 includes control logic that uses inputs and outputs from building subsystems to provide greater comfort and energy savings relative to the comfort and energy savings that separate subsystems could provide alone. For example, integrated control layer 218 can be configured to use an input from a first subsystem to make an energy-saving control decision for a second subsystem. Results of these decisions can be communicated back to building subsystem integration layer 220.

Integrated control layer 218 is shown to be logically below demand response layer 214. Integrated control layer 218 can be configured to enhance the effectiveness of demand response layer 214 by enabling building subsystems 228 and their respective control loops to be controlled in coordination with demand response layer 214. This configuration can reduce disruptive demand response behavior relative to conventional systems. For example, integrated control layer 218 can be configured to assure that a demand response-driven upward adjustment to the setpoint for chilled water temperature (or another component that directly or indirectly affects temperature) does not result in an increase in fan energy (or other energy used to cool a space) that would result in greater total building energy use than was saved at the chiller.

Integrated control layer 218 can be configured to provide feedback to demand response layer 214 so that demand response layer 214 checks that constraints (e.g., temperature, lighting levels, etc.) are properly maintained even while demanded load shedding is in progress. The constraints can also include setpoint or sensed boundaries relating to safety, equipment operating limits and performance, comfort, fire codes, electrical codes, energy codes, and the like. Integrated control layer 218 is also logically below fault detection and diagnostics layer 216 and automated measurement and validation layer 212. Integrated control layer 218 can be configured to provide calculated inputs (e.g., aggregations) to these higher levels based on outputs from more than one building subsystem.

Automated measurement and validation (AM&V) layer 212 can be configured to verify that control strategies commanded by integrated control layer 218 or demand response layer 214 are working properly (e.g., using data aggregated by AM&V layer 212, integrated control layer 218, building subsystem integration layer 220, FDD layer 216, or otherwise). The calculations made by AM&V layer 212 can be based on building system energy models and/or equipment models for individual BAS devices or subsystems. For example, AM&V layer 212 can compare a model-predicted output with an actual output from building subsystems 228 to determine an accuracy of the model.

Fault detection and diagnostics (FDD) layer 216 can be configured to provide on-going fault detection for building subsystems 228, building subsystem devices (i.e., building equipment), and control algorithms used by demand response layer 214 and integrated control layer 218. FDD layer 216 can receive data inputs from integrated control layer 218, directly from one or more building subsystems or devices, or from another data source. FDD layer 216 can automatically diagnose and respond to detected faults. The responses to detected or diagnosed faults can include providing an alarm message to a user, a maintenance scheduling system, or a control algorithm configured to attempt to repair the fault or to work-around the fault.

FDD layer 216 can be configured to output a specific identification of the faulty component or cause of the fault (e.g., loose damper linkage) using detailed subsystem inputs available at building subsystem integration layer 220. In other exemplary embodiments, FDD layer 216 is configured to provide “fault” events to integrated control layer 218 which executes control strategies and policies in response to the received fault events. According to an exemplary embodiment, FDD layer 216 (or a policy executed by an integrated control engine or business rules engine) can shut-down systems or direct control activities around faulty devices or systems to reduce energy waste, extend equipment life, or assure proper control response.

FDD layer 216 can be configured to store or access a variety of different system data stores (or data points for live data). FDD layer 216 can use some content of the data stores to identify faults at the equipment level (e.g., specific chiller, specific AHU, specific terminal unit, etc.) and other content to identify faults at component or subsystem levels. For example, building subsystems 228 can generate temporal (i.e., time-series) data indicating the performance of BAS 200 and the various components thereof. The data generated by building subsystems 228 can include measured or calculated values that exhibit statistical characteristics and provide information about how the corresponding system or process (e.g., a temperature control process, a flow control process, etc.) is performing in terms of error from its setpoint. These processes can be examined by FDD layer 216 to expose when the system begins to degrade in performance and alarm a user to repair the fault before it becomes more severe.

Referring now to FIG. 3, a schematic drawing of a floor 300 of a school building is shown, according to an exemplary embodiment. The floor 300 can be a floor including multiple classrooms 302-306 connected by a hallway 308. A school building system can be configured to utilize attendance data of the school to determine infectious disease risk data for the school building, for individual spaces of the school building (e.g., the classrooms 302-306 or the hallway 308), for students, teachers, or staff of the school building, for the school building itself, etc.

The attendance data can indicate whether students, teachers, or staff have attended school on a particular day or days, have attended classes or meetings on a particular day or days, have shown up to teach school on a particular day or days, etc. If a person is absent, this may indicate that the person is potentially sick with an infectious disease. If a person is absent, the school building system can set their infectious disease risk score to a high level, e.g., 80-100%. Furthermore, based on school schedule data, classroom layout data, school layout data, etc. the school building system can determine infectious disease risk scores for various spaces and/or other individuals of the school building.

For example, if the classroom 304 has five students absent on a particular day, indicated by attendance data and classroom schedule data, the building system can determine an infectious disease risk score for the classroom 304 based on the number of students that were absent, e.g., five. The higher the number of students absent, the higher the building school system can determine the infectious disease risk score for the classroom. The number of students absent from a class or classes of the classroom 304 can be mapped to risk scores for the classroom 304. Furthermore, the longer the length of time that the absent students spent time in the classroom 304 over some timer period, the higher the infections disease risk score for the classroom.

Furthermore, the school building system can determine an infectious disease risk score for present students of the classroom 304 based on the number of students absent from the class. This may affect the present students because they were previously in contact with the absent students that are potentially sick, e.g., they attended class together on a previous day. The number of contacts between a present students and absent students over a historical window (e.g., for a previous day, week, or month) can be used by the school building system to determine an infectious disease risk score for the student. In some embodiments, the class schedule for a day or week is used for the student to determine the number of previous interactions between potentially infected students that are absent from class and present students that are attending class. The number of interactions can be used to determine a risk score for the students, e.g., 0-2 interactions could be mapped to a low risk score, 3-5 could be mapped to a medium risk score, and 6 or more could be mapped to a high risk score.

Furthermore, the length of time of each interaction can influence the risk score for students and/or spaces. If a potentially infected student that is absent from class was in classroom 302 for a half hour the day before the student is absent and the absent student was in the classroom 306 for an hour and a half the day before the student was absent, the school building system can determine a low risk score for the classroom 302 and a high risk score for the classroom 306. Furthermore, the infectious disease risk score for students of the classroom 306 can be set higher than the students of the classroom 302 because the students of the classroom 306 were exposed to the potentially infected student for a longer time period.

In some embodiments, the length of time of an interaction between students and/or classrooms can be treated as one or multiple interactions. For example, if two students are in a classroom together for a half hour, this could be treated as a single interaction between the two students and between the classroom. If the two students are in the classroom for an hour, this could be treated as two interactions because of the extended duration. If the two students are in the classroom for an hour and a half, this could be treated as three separate interactions because the students were in contact for such an extended duration.

The historical daily and/or weekly number of interactions between a student and potentially infected students and/or between a classroom and potentially infected students can then be mapped by the building school system to infectious disease risk score levels. For example, 0-5 interactions could be mapped to a low infectious disease risk level, 6-10 interactions could be mapped to a medium infectious disease risk level, while 11 or more interactions could be mapped to a high infectious disease risk level.

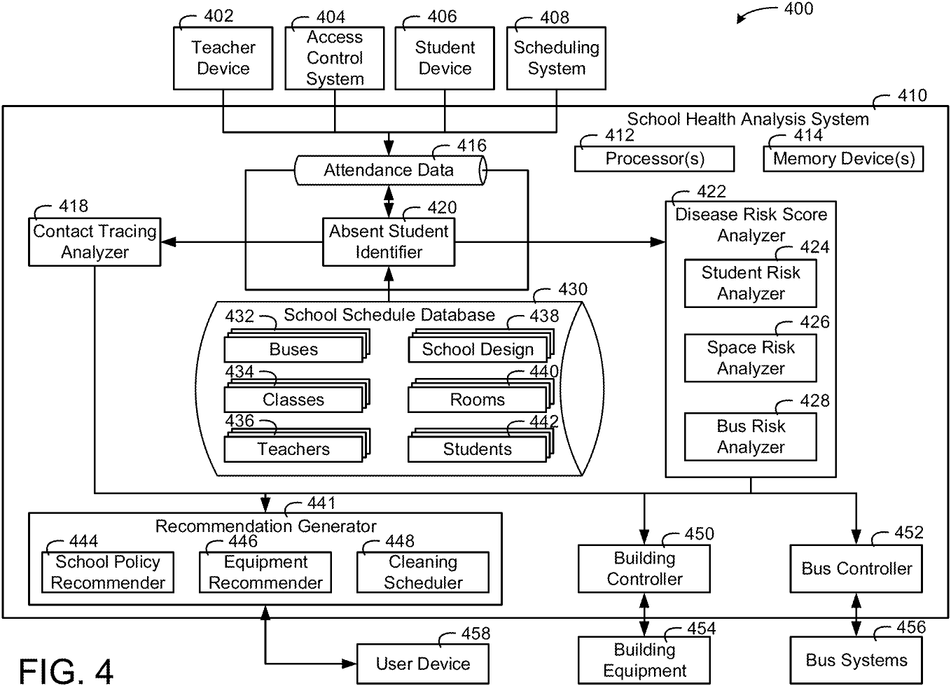

Referring now to FIG. 4, a system 400 including a school health analysis system 410 that can determine disease risk scores for the school building based on attendance data of the school building is shown, according to an exemplary embodiment. The system 410 can include processors 412 and memory devices 414. The processors 412 and/or memory devices 414 can be devices of one or multiple servers, computer systems, cloud systems, etc. The processors 412 can be general purpose or specific purpose processors, application specific integrated circuits (ASICs), one or more field programmable gate arrays (FPGAs), a group of processing components, and/or other suitable processing component. The processors 412 may be configured to execute computer code and/or instructions stored in the memory devices 414 or received from other computer readable media (e.g., CDROM, network storage, a remote server, etc.).

The memory devices 414 can include one or more devices (e.g., memory units, memory devices, storage devices, etc.) for storing data and/or computer code for completing and/or facilitating the various processes described in the present disclosure. The memory devices 414 can include random access memory (RAM), read-only memory (ROM), hard drive storage, temporary storage, non-volatile memory, flash memory, optical memory, or any other suitable memory for storing software objects and/or computer instructions. The memory devices 414 can include database components, object code components, script components, or any other type of information structure for supporting the various activities and information structures described in the present disclosure. The memory devices 414 can be communicably connected to the processors 412 and can include computer code for executing (e.g., by the processors) one or more of the processors 412 described herein.

The system 410 can be connected to external systems that can be used to collect attendance data 416. The external systems could be a teacher device 402. The teacher device 402 could be a laptop, a smartphone, a desktop computer, etc. that the teacher provides student attendance data to the system 410. The external systems can include an access control system 404. The access control system 404 can control badge access, identifier card access, biometric based access, etc. to the school building. Access data of the access control system 404 can indicate which students, teachers, or staff have accessed the school building and are thus present at the school building.

The external systems include student devices. The student devices 406 can include smartphones, laptops, tablets, etc. A student can enter an indication into their student device 406 to indicate whether they will be absent or present at classes on a particular day. In some embodiments, the student device may log onto a school network and/or connect to an access point located at the school building. Responsive to detecting that the student device 406 is logged into the network, the system 410 can determine that the student is present. In some embodiments, the student devices 406 can be a phone, tag, or identification card that is tracked by wireless systems (e.g., a network system, a scanner system, a beacon system, etc.) of the school building and can provide a location of the user within the school building. The student device 406 can be a school issued device, a device that runs software for the school building, or a private device of a student. In some embodiments, the tracking is location tracking, e.g., where in the building the students are located. In some embodiments, the tracking is on a general level and only determine whether the student is at the school building or not. In some embodiments, the tracking is only implemented when the student is in the school building and not when the student is outside the school building. The tracking can be real-time tracking performed by an on-premises location tracking system (e.g., a Wi-Fi triangulation system, an ad-hoc beacon based system, etc.). Examples of tracking systems are found in U.S. Pat. Application No. 15/812,260 filed November 14^{th}, 2017 and U.S. Pat. Application No. 17/220,795 filed April 1^{st} 2021, the entirety of each of these patent applications is incorporated by reference herein.

The external systems can include a scheduling system 408. The scheduling system 408 can be a scheduling system for the school building that receives indications such as sick days, parental leave notices, etc. In some embodiments, the system 410 can analyze the scheduling data of the scheduling system 408 to identify whether one or more students, teachers, or staff are absent from the building due to using a sick day, having a parental note indicating that the student is stick, etc. In some embodiments, the scheduling data of the scheduling system 408 can include natural language information describing why person is present or absent from the school building. The system 410 can analyze the data to determine if the person is absent because they are sick.

The attendance data 416, in some embodiments includes a language description of a reason why a student, teacher, or staff member is absent from school. The language description could include one or more characters, symbols, words, phrases, acronyms, etc. The language description could be “Vacation,” “Sick Day,” “Attending family event,” etc. The system 410 can be configured to analyze the language description to categorize the individual being absent as a risk to infection or a non-risk to infection. For example, if the description is “Not feeling well” or “Sick Day” as an infection risk but “Vacation” or “Traveling” as not pertaining to an infection risk. Examples of natural language processing that can be applied by the system 410 can be found in U.S. Pat. Application No. 16/143,256 filed September 26^{th}, 2018, the entirety of which is incorporated by reference herein.

The data and/or determinations collected from the systems and devices 402-408 can be stored in a database for attendance data 416 which can store all of the attendance related information collected from the systems and devices 402-408. The attendance data 416 may in some embodiments expressly identify each student that is present, each student that is absent, each teacher that is present, each teacher that is absent, each staff member that is present, each staff member that is absent, etc. However, in some embodiments, the absent students are not expressly identified in the attendance data 416.

The system 410 includes an absent student identifier 420. The absent student identifier 420 can identify, based on the attendance data 416 and schedule data of the school schedule database 430, which students are absent. In some embodiments, the absent student identifier 420 can compare a list of present students for a class, class period, and/or day against a schedule of students expected to be present for the class, class period, and/or day. Any students that should be present but are not can be identified as absent students by the absent student identifier 420. In some embodiments, the absent student identifier 420 can aggregate indications of absent students from the attendance data 416.

In some embodiments, the disease risk score analyzer 422 can be based on geographic data and/or infection risk levels of the geographic data. For example, the analyzer 422 could communicate with a third party health data source, e.g., a private company system and/or a health department system, e.g., the Center For Disease Control (CDC), the World Health Organization (WHO), etc. The third party health system can provide the analyzer 422 with indications of number of active or historical cases of individuals infected with the infectious disease. The disease risk score analyzer 422 can calculate a baseline risk level for students, spaces, and/or buses based on the geographic risk data, e.g., the number of cases in a neighborhood of the school building, a county of the school building, a state of the school building, and/or the country that the school building is located in.

The system 410 includes a disease risk analyzer 422. The disease risk analyzer 422 can be configured to determine disease risk levels for students, spaces, and/or buses. The disease risk analyzer 422 can determine the disease risk level based on the absentee data received from the identifier 420 and/or a database storing the attendance data 416. In some embodiments, the disease risk analyzer 422 can determine risk levels based on other pieces of building information, e.g., temperature, humidity CO2 levels, etc. In some embodiments, the risk levels are based on how well an individual and/or a group of individuals wear a mask, use sanitization, practice social distancing, etc. Examples of disease related risk level determinations that the disease risk analyzer 422 can perform can be found in U.S. Pat. Application No. 17/220,795 filed April 1^{st}, 2021, U.S. Pat. Application No. 17/067,211 filed October 9^{th}, 2020, U.S. Pat. Application No. 17/013,273 filed September 4^{th}, 2020, U.S. Pat. Application No. 17/354583 filed June 22^{nd}, 2021, U.S. Pat. Application No. 17/354565 filed June 22^{nd}, 2021, U.S. Publication No. 16/927,759 filed July 13^{th}, 2020, and U.S. Publication No. 16/927,318 filed July 13^{th}, 2020. The entirety of each of these patent applications is incorporated by reference herein.

In some embodiments, the attendance data 416 includes an identify of each student (e.g., absent students, present students). In some cases, the attendance data 416 (or alternatively the school schedule database 430) can indicate the locations that the students are present at or are absent from.

The school schedule database 430 can store information tracking the schedules of various teachers, students, staff, vehicles (e.g., buses), class rooms, etc. The schedule can indicate which teachers 436 and/or which students 442 rode which buses 432, are part of which classes 434, and/or what rooms 440 each of the classes 434 is assigned. The schedule of the school schedule database 430 could further indicate the length of each of the classes 434. The schedule data of the school schedule database 430 can be provided to the absent student identifier 420 for identifying which students should normally be present at a class so that an attendance record can be compared against the expected students to identify the absent students. The schedule data of the school schedule database 430 can also be provided to the contact tracing analyzer 418. The contact tracing analyzer 418 can analyze the schedule activity to identify potentially students or teachers infected and/or potential infectors.

In some embodiments, the database 430 includes school design data 438. The school design data 438 can indicate hallway sizes, space sizes, equipment for the spaces, airflow through the school building, etc. In some embodiments, the disease risk score analyzer 422 can determine an infection risk level based on the school design data 438, e.g., based on a physical design and/or construction of the school building and equipment operating settings of equipment of the school building. For example, space disease risk scores for spaces with disinfectant light systems may be lower than similar spaces without he disinfectant light systems.

The disease risk score analyzer 422 can include a student risk analyzer 424. The analyzer 424 can be configured to determine risk scores for contracting an infectious disease for individual students (or teachers and/or staff). The student risk analyzer 424 could identify, over a historical time period, how many absent students a particular student interacted with (e.g., went to the same class, club meeting, band practice, etc.). The number of interactions can be the risk score and/or can be used by the risk analyzer 424 to determine a risk score. In some embodiments, the risk score for individuals can be based on a length of time that a first occupant is in a same space (or within a particular distance) from a second occupant. For example, if the second occupant is potentially sick, e.g., is absent, and the first occupant and the second occupant have had three hours of shared classroom time. The student risk analyzer 424 can use the school schedule database 430 to determine the shared classroom time and can generate the score based on the shared classroom time.

The disease risk score analyzer 422 can include a space risk analyzer 426. The space risk analyzer 426 can determine risk levels for a particular classroom, hallway, school building, etc. The space risk analyzer 426 can determine the space risk scores based on the school schedule data of the database 430, indications of absent students determined by the absent student identifier 420, and/or the attendance data 416. For example, the space risk analyzer 426 could identify events occurring within a space, e.g., a class session, a band practice, a club meeting, etc. based on the school schedule data of the database 430. The events can occur at certain times with certain students and/or teachers. The space risk analyzer 426 could identify a number of events where an absent student was previously present, e.g., three events in the past week included one, four, and ten infected students respectively. The space risk analyzer 426 can generate the space infection risk score based on the number of events that an absent student was previously present at and/or the number of absent students from events at the particular space.

In some embodiments, the space infection risk level of various spaces determined by the space risk analyzer 426 can be provided to a recommendation generator 441. The recommendation generator 441 can generate recommendations via a school policy recommender 444, an equipment recommender 446, and/or a cleaning scheduler 448. For example, a space with a risk level greater than a particular level could have a cleaning (e.g., disinfectant cleaning) scheduled and/or recommended for the particular space. The equipment recommender 446 could recommend a control algorithm (e.g., a high air change rate) that would operate equipment for the space to reduce the likelihood of the spread of an infectious disease. Furthermore, the equipment recommender 446 could recommend that filtration equipment (e.g., portable filtration equipment) be installed in the high risk space.

In some embodiments, the disease risk score analyzer 422 can determine that a student that has an infectious disease risk level above a particular amount is a potentially infected individual. In some embodiments, the space risk analyzer 426, the bus risk analyzer 428, and/or the student risk analyzer 424 can determine risk levels based not only on absent students (who are potentially infected) but also based on individuals who have an infection risk level above a particular level.

The bus risk analyzer 428 can be configured to determine infection risk levels for busses and/or other transportation vehicles of the school building. Furthermore, the bus risk analyzer 428 can be configured to determine infection risk levels for riders of the buses and/or other transportation vehicles, students, teachers, staff, bus drivers, etc. For example, the bus risk analyzer 428 could be configured to determine the number of potentially infected students (e.g., students that are absent or have a risk level over a particular amount) that have ridden a particular bus and/or the length of time that the potentially infected students were on the bus.

In some embodiments, the bus risk analyzer 428 can calculate and/or adjust student risk levels based on the risk level of the bus that the students have ridden on. For example, a risk level of a student could be increased by a value proportional to the risk level of the bus that the student rode on and/or based on a length of time that the student was on the bus. In some embodiments, a student could be assigned the same risk level as the bus (if the students risk level is less than the bus) if the student rides on the bus for at least a particular length of time.

In some embodiments, the bus risk analyzer 428 can determine a group of students that ride the busses to get to school, to get to a school event, to get to a sporting event, etc. based on the data of the school schedule database 430. The bus risk analyzer 428 can identify one or more individuals of the group that are potentially infected, e.g., are absent students and/or have risk levels greater than a particular amount. The bus risk analyzer 428 can determine a risk level for each student in the group of students, and/or an adjustment to the risk levels for the group of students, based on the number of potentially infected students in the group of students.

In some embodiments, a bus controller 452 can operate (or command) bus systems 456 of various buses based on the risk levels of the various buses and/or the risk levels of students riding on the various buses. In some embodiments, the bus controller 452 can communicate (e.g., via cellular communication, Wi-Fi, Bluetooth, etc.) with the bus systems 456 and cause the bus systems 456 to increase outdoor air, increase ventilation, increase filtration, etc. to reduce the likelihood of the spread of the infectious disease in the bus. In some embodiments, the control settings for the bus systems 456 could be based on the infection level of the bus and/or students of the bus. In some embodiments, the cleaning scheduler 448 could schedule cleaning for a bus based on the infection level of the bus and/or students riding on the bus.

The building controller 450 can be configured to operate the building equipment 454 to reduce a student infection level and/or a space infection level, etc. The building controller 450 could be configured to operate disinfectant light in spaces, increase outdoor air, increase filtration for spaces, increase temperatures and/or humidities to levels that kill infectious disease, etc. Examples of building control that reduces the spread of disease can be found in U.S. Pat. Application No. 17/013,273 filed September 4^{th}, 2020, the entirety of which is incorporated by reference herein. In some embodiments, the building controller 450 can operate spaces based on the space risk levels of various spaces. For example, the ventilation of a space could be set to a particular level based on the risk level of the space, e.g., the higher the risk level the higher the ventilation rate. In some embodiments, the building controller 450 takes actions to reduce the spread of an infectious disease in a space if the risk level of the space is greater than a particular amount.

The contact tracing analyzer 418 can be configured to perform contact tracing to identity potentially impacted students that have been impacted by an infected or potentially infected student. In some embodiments, the contact tracing analyzer 418 can receive indications of absent students from the absent student identifier 420 that the absent student identifier 420 determines based on the attendance data 416. The contact tracing analyzer 418 can identify the potentially impacted students by analyzer schedule data of the school schedule database 430. The contact tracing analyzer 418 can identifier interaction events between students. The events can indicate that, historically, certain students had direct or indirect interaction with the one or more absent students. The students who interacted with the absent students can be identified as potentially impacted (e.g., potentially infected) by the contact tracing analyzer 418. In some embodiments, the contact tracing analyzer 418 can identify that potentially impacted students were in a same space as the one or more absent students. The contact tracing analyzer 418 can perform a cluster analysis where clusters of students are analyzed to identify what students contracted the infectious disease and what event or individual caused the students to contract the disease.

The contact tracing analyzer 418 can be configured to identify potentially impacted students of the school building that may have contracted a disease from teachers and/or staff. The absent student identifier 420 can further identify absent teachers and/or staff. The absence of teachers and/or staff may indicate that the teachers and/or staff have contracted an infectious disease. The contact tracing analyzer 418 can identify that one or more students had direct or indirect interaction with the absent staff and/or teacher in the past. For example, the contact tracing analyzer 418 could identify that a student went to tutoring to meet with a tutor. However, the analyzer 418 can identify that the tutor is now absent, indicating that the student may have been in contact with an infected individual (the tutor). The contact tracing analyzer 418 could identify that a teacher is now absent from a class that the teacher taught and that the students of the class are now potentially infected.

The recommendation generator 441 can be configured to generate recommendations for reducing the spread of an infectious disease in the school building and/or reduce the infection risk level of a student, space, and/or bus. The recommendation generator 441 can include a school policy recommender 444, an equipment recommender 446, and/or a cleaning scheduler 448.

The school policy recommender 444 can be configured to determine policy recommendations for the school building. The policy recommendations can recommend an infectious disease prevention policy based on a student disease risk level, a space disease risk level, a bus disease risk level, an overall school disease risk level (e.g., an average of all risk levels of all spaces, students, and/or buses of the school building), etc. In some embodiments, the policy could be a policy that students, staff, and/or teachers (or specific groups of students, staff, and/or teachers) wear a mask while at the school building. The policy could be a remote school policy indicating that students, staff, and/or teachers (or specific groups of students, staff, and/or teachers) attend classes remotely, e.g., via their computers in their own homes. In some embodiments, the policy could be a social distancing policy.

In some embodiments, the policy recommender 444 can recommend policies based on the infection risk levels of various students, teachers, and/or staff. For example, the recommender 444 could identify a policy and/or policy set for individuals with risk levels greater than a particular level. The policy for the group could be to wear masks, attend class remotely, social distance, etc.

The equipment recommender 446 can recommend temporary and/or permanent equipment installation and/or operation (e.g., operating settings, control algorithms, etc.) for the school building and/or various spaces of the school building. For example, the recommendations generated by the equipment recommender 446 could be a recommendation to perform a retrofit of equipment for the school building and/or spaces of the school building. The retrofit could further be changing the layout and/or structure of the school building and/or spaces of the school building, e.g., adding more windows that can open and ventilate classrooms, widening hallways to make it easier for individuals to social distance, etc.

The equipment recommender 446 can generate recommendations for adding or retrofitting equipment of a school. In some embodiments, retrofitting the equipment could include installing high performance filters in air handling equipment, installing disinfectant kill tunnels in air ducts of the building, etc. The equipment could be temporary equipment, e.g., a portable filtration system that could be added to a classroom, auditorium, etc. The recommender 446 could generate a recommendation to add portable filtration equipment to spaces of the school building that have an infection risk level greater than a particular amount.

The cleaning scheduler 448 can be configured to schedule cleaning (or recommend cleaning) for various spaces of the school building. The cleaning recommended can be for various levels of cleaning, e.g., basic cleaning, moderate cleaning, deep cleaning, chemical disinfection, fumigation, etc. The scheduler 448 can schedule cleaning for various spaces based on the school building based on the infection risk levels for the various spaces. For example, once the infection level reaches a particular amount, the cleaning scheduler can schedule cleaning for the space. The level of cleaning for the space can be selected based on the risk level for the space. In some embodiments, the cleaning scheduler 446 can push scheduling appointments to cleaning personnel. In some embodiments, the cleaning scheduler 446 can communicate with robotic cleaning equipment, e.g., robots, disinfectant equipment installed within spaces, drones, etc.

The system includes a user device 458. The user device 458 can be a smartphone, a laptop computer, a desktop computer, a tablet computer, etc. The user device 458 can be the same as or similar to the client devices 248. The user device 458 can display the recommendations of the recommendation generator 441 in a user interface of the user. The user can accept and/or reject the recommendations via the user device 458.

In some embodiments, the recommendation generator 441 can generate recommendations for schools other than the school building that the system 410 performs the analysis for. The recommendation generator 441 can compare schools against each other and determine which recommendations for one school would be applicable for another school. The generator 441 can use risk levels calculated for a first school to determine recommendations for a second school that is within a particular distance from the first school or in a similar geographic region as the first school. This can enable the generator 441 to generate recommendations for a school even when the data to perform the risk analysis described in FIG. 4 is unavailable.

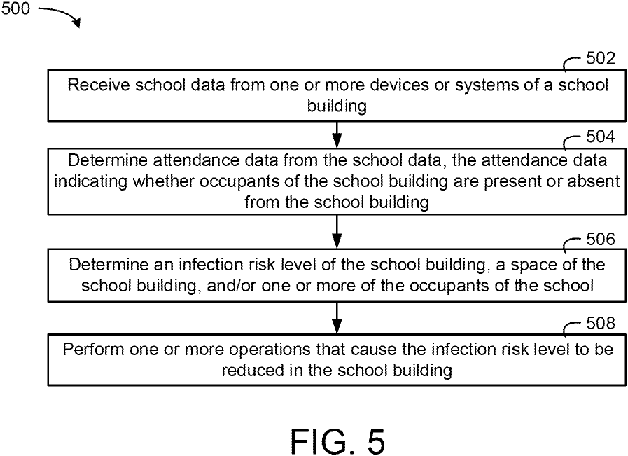

Referring now to FIG. 5, a flow diagram of a process 500 of determining an infection risk level based on attendance data for the school building is shown, according to an exemplary embodiment. The process 500 can be performed by the school health analysis system 410, in some embodiments. Any computing system or device described herein can be configured to perform the process 500.

In step 502, the system 410 can receive school data from one or more devices or systems of the school building. The system 410 can receive school data that indicates the attendance of students, teachers, and/or staff, e.g., the attendance data 416. The data can be received from various systems and devices, e.g., the teacher device 402, the access control system 404, the student device 406, and/or the scheduling system 408.

In step 504, the system 410 can determine attendance data from the received school data in the step 502. The attendance data determined in the step 504 can be the attendance data 416. The attendance data 416 can indicate students, teachers, and/or staff that are and are not present at school. The absent student identifier 420 can use the school data and the school schedule data of the school schedule database 430 to determine which students are present or absent from the school on a particular day and/or days.

In step 506, the system 410 determines an infection risk level of the school building, a space of the school building, and/or one or more occupants of the school building (e.g., students, teachers, staff, etc.). The disease risk score analyzer 422 can use the attendance data 416, indications of absent students determined by the absent student identifier 420, and/or schedule data of the school schedule database 430 to determine the risk levels.

In step 508, the system 410 performs one or more operations for causing the infection risk level to be reduced in the school building. For example, in some embodiments, the operations could be updating the operating settings of the building equipment 454 of the school building to cause the building equipment 454 to operate in a manner that reduces the spread of an infectious disease in the building (e.g., activate disinfectant light, increase outdoor air, increase ventilation, etc.). In some embodiments, the operations can include recommending and/or setting a school policy, e.g., a mask policy, a remote learning policy, etc. In some embodiments, the operations can be implementing and/or scheduling cleaning and/or disinfection.

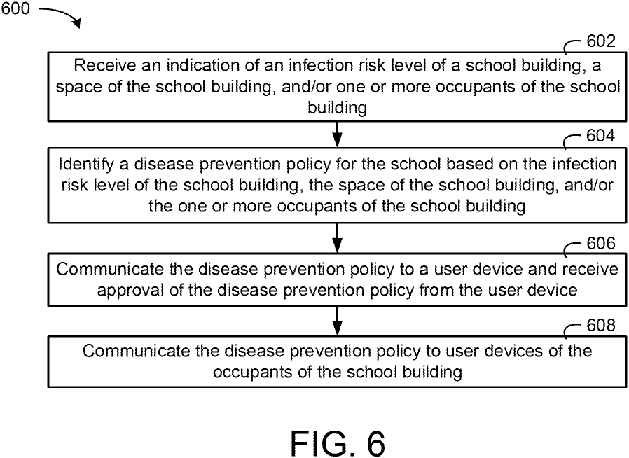

Referring now to FIG. 6, is a flow diagram of a process 600 of using an infection risk level to determine and set a disease prevention policy for the school building is shown, according to an exemplary embodiment. The process 600 can be performed by the school health analysis system 410, in some embodiments. Any computing system or device described herein can be configured to perform the process 600.

In step 602, the system 410 receives an indication of an infection risk level of the school building, a space of the school building, and/or the occupants of the school building. The infection risk levels can be determined by the disease risk score analyzer 422 (e.g., the student risk analyzer 424, the space risk analyzer 426, and/or the bus risk analyzer 428). In some embodiments, the infection risk levels are received from another system or device.

In step 604, the system 410 identifies a disease prevention policy for the school building based on the infection risk levels. The disease prevention policy can be based on the level of the infectious disease risk, e.g., whether it is within certain ranges of values. Each range of values can correspond with a particular policy and/or policy set. The policies could include a remote learning policy where all students and/or staff (or certain groups of students and/or staff) attend classes and/or meetings remotely. The policies could include mask policy where all teachers and/or students (or certain potentially infected students) wear masks.

In step 606, the system 410 communicates he disease prevention policy to a user device, e.g., a user devices of a principal, school board member(s), teachers union members, etc. The system 410 can receive approval of the policy from the user device. In step 608, the system 410 can communicate the disease prevention policy to user devices of occupants of the school building. For example, a mass email, mass text, individual email, individual text, mass message, individual message, etc. can be communicated to the devices of the teachers, students, and/or staff to notify the teachers, students, and/or staff of the new protocol.

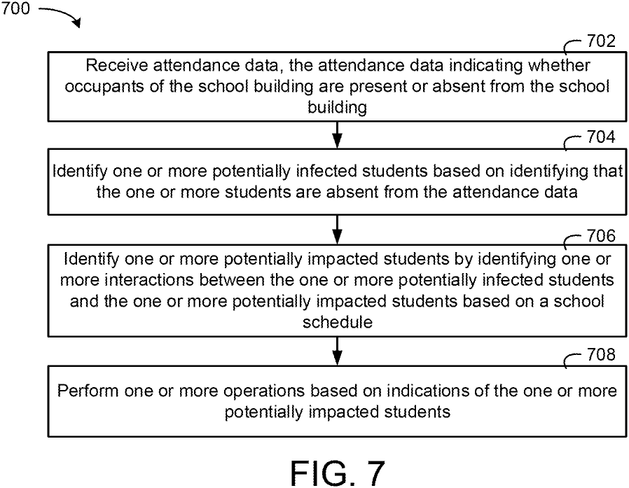

Referring now to FIG. 7, a process 700 of performing contact tracing for the school building based on attendance data for the school building and a schedule of the school building is shown, according to an exemplary embodiment. The process 700 can be performed by the school health analysis system 410, in some embodiments. The process 700 can be performed by the contact tracing analyzer 418, in some embodiments. Any computing system or device described herein can be configured to perform the process 700.

In step 702, the system 410 receives attendance data, the attendance data indicating whether occupants of the school building are present or absent from the school building, e.g., are present or absent from school on a particular day and/or are present or absent from a class and/or classes. The attendance data can be the attendance data 416 and/or indications of absent students identified by the identifier 420.

In step 704, the system 410 identifies one or more potentially infected students based on the indications of the one or more students that are absent from class based on the attendance data received in step 702. For example, the identifier 420 could compare the attendance data 416 against the schedule data of the school schedule database 430 to identify the absent students.

In step 706, the system 410 identifies one or more potentially impacted students by identifying one or more interactions between the one or more potentially infected students and the one or more potentially impacted students based on a school schedule. The system 410 identifies the potentially impacted students by identifying, based on the data of the database 430, whether the impacted students and the infected students shared the same classes, sporting events, club events, etc.

In step 708, based on the indications of the impacted students, the system 410 can perform operations that reduce the spread of an infectious disease. For example, the system 410 can operate the building equipment 454 to reduce the spread of an infectious disease in the school building, e.g., increase ventilation, operate at temperatures and/or humidities that reduce the spread of a disease, etc.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

The present disclosure contemplates methods, systems and program products on any machine-readable media for accomplishing various operations. The embodiments of the present disclosure can be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures show a specific order of method steps, the order of the steps may differ from what is depicted. Also two or more steps can be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

What is claimed:

1. A building system for a school building, the building system including one or more memory devices storing instructions thereon that, when executed by the one or more processors, cause the one or more processors to:
receive attendance data indicating whether a plurality of occupants of the school building are present or absent from the school building;
determine, based on the attendance data, an infection risk level of at least one of the school building, a space of the school building, or one or more of the plurality of occupants to being infected with an infectious disease present in a population; and
perform one or more operations for causing the infection risk level to be reduced in the school building.

2. The building system of claim 1, wherein the attendance data includes identities of a plurality of students and class locations of classes that the plurality of students are present at or absent from.

3. The building system of claim 1, wherein the instructions cause the one or more processors to:
receive class schedules of a plurality of classes of the school building, the class schedules indicating the plurality of classes, identities of students associated to the plurality of classes, room assignments of the plurality of classes, a class length of the plurality of classes, and teachers of the plurality of classes; and
determine, based on the attendance data and the class schedules, the infection risk level of the at least one of the space or the plurality of occupants.

4. The building system of claim 1, wherein the plurality of occupants comprise a plurality of students, and wherein the instructions cause the one or more processors to:
determine a space infection level for the space of the school building based on the attendance data and a school schedule indicating events occurring in certain spaces of the school building at certain times with certain students of the plurality of students; and
implement, responsive to determining the space infection level of the space is above a predefined amount, at least one of scheduling cleaning for the space, implementing updated environmental control of the space, or recommending additional filtration equipment for the space.

5. The building system of claim 1, wherein the instructions cause the one or more processors to:
determine, based on the attendance data, the infection risk level of a plurality of groups of students of the school building; and
recommend one or more first groups of students of the plurality of groups of students attend in person class and one or more second groups of students of the plurality of groups of students attend remote class based on the infection risk level of the plurality of groups of students.

6. The building system of claim 1, wherein the infectious disease is a human transmitted disease transmitted between the plurality of occupants via air, physical contact with surfaces, and/or physical contact between the plurality of occupants.

7. The building system of claim 1, wherein the one or more operations include operating one or more pieces of equipment in the school building to reduce the infection risk level.

8. The building system of claim 1, wherein the one or more operations include generating a recommendation for adding or retrofitting equipment of the school building, wherein the added or retrofit equipment operate to reduce the infection risk level.

9. The building system of claim 1, wherein the one or more operations include scheduling cleaning in one or more areas of the school building.

10. The building system of claim 1, wherein the instructions cause the one or more processors to:
determine one or more recommendations for one or more second school buildings based on the infection risk level of the school building.

11. The building system of claim 1, wherein the instructions cause the one or more processors to determine the infection risk level for a first occupant based on an amount of time the first occupant spent in a same space as a second occupant, the amount of time determined using the attendance data.

12. The building system of claim 1, wherein the one or more operations comprise either deploying or recommending deployment of a portable filtration device.

13. The building system of claim 1, wherein the plurality of occupants comprise a plurality of students, and wherein the instructions cause the one or more processors to:
perform contact tracing to identify one or more potentially impacted students of the plurality of students by:
identifying one or more absent students based on the attendance data; and
identifying the one or more potentially impacted students based on the one or more absent students and a school schedule, wherein the school schedule indicates interaction events between the plurality of students and indicates that the one or more potentially impacted students had direct or indirect interaction with the one or more absent students.

14. The building system of claim 13, wherein the school schedule indicates that the one or more potentially impacted students were in a same space of the school building as the one or more absent students.

15. The building system of claim 1, wherein the instructions cause the one or more processors to determine the infection risk level further based on a physical design and/or construction of the school building and equipment operating settings of equipment of the school building.

16. The building system of claim 15, wherein the instructions cause the one or more processors to determine the infection risk level further based on third party health data of a geographic region where the school building is located.

17. The building system of claim 1, wherein the plurality of occupants comprise a plurality of students, and wherein the instructions cause the one or more processors to:
determine one or more potentially infected students of the plurality of students based on the attendance data;
determine a plurality of groups of students that ride school busses;
identify one or more groups of the plurality of groups of students that include the one or more potentially infected students; and
determine the infection risk level based on the one or more groups of the plurality of groups of students that include the one or more potentially infected students.

18. The building system of claim 17, wherein the instructions cause the one or more processors to:
identify one or more school buses of the school busses that the one or more potentially infected students have ridden on or are riding on; and
perform at least one of environmental control of the one or more school buses or scheduling cleaning for the one or more school buses.

19. The building system of claim 1, wherein the one or more operations include determining an infectious disease prevention policy recommendation for the school building using the infection risk level.

20. The building system of claim 19, wherein the infectious disease prevention policy recommendation is at least one of a mask requirement, a remote school policy, an in person policy, or a social distancing requirement.

21. The building system of claim 1, wherein the plurality of occupants comprise a plurality of students and a plurality of staff members, the plurality of staff members comprising a plurality of teachers, and wherein the instructions cause the one or more processors to:
perform contact tracing to identify one or more potentially impacted students of the plurality of students by:
identifying an absent staff member based on the attendance data; and
identifying the one or more potentially impacted students based on the absent staff member and a school schedule, wherein the school schedule indicates interaction events between the one or more potentially impacted students and the absent staff member and indicates that the one or more potentially impacted students had direct or indirect interaction with the absent staff member.

22. The building system of claim 21, wherein the instructions cause the one or more processors to identify the one or more potentially impacted students using the attendance data and the school schedule, and wherein the school schedule comprises data indicating classes that the one or more potentially impacted students attended and data indicating classes that the absent staff member taught.

23. A method comprising:
receiving, by a processing circuit, attendance data indicating whether a plurality of occupants of a school building are present or absent from the school building;
determining, by the processing circuit, based on the attendance data, an infection risk level of at least one of the school building, a space of the school building, or one or more of the plurality of occupants to being infected with an infectious disease present in a population; and
performing, by the processing circuit, one or more operations for causing the infection risk level to be reduced in the school building.

24. The method of claim 23, wherein the attendance data includes identities of a plurality of students and class locations of classes that the plurality of students are present at or absent from.

25. The method of claim 23, further comprising:
receiving, by the processing circuit, class schedules of a plurality of classes of the school building, the class schedules indicating the plurality of classes, identities of students associated to the plurality of classes, room assignments of the plurality of classes, a class length of the plurality of classes, and teachers of the plurality of classes; and
determining, by the processing circuit, based on the attendance data and the class schedules, the infection risk level of the at least one of the space or the plurality of occupants.

26. The method of claim 23, wherein the plurality of occupants comprise a plurality of students;
wherein the method further comprises:
determining, by the processing circuit, a space infection level for the space of the school building based on the attendance data and a school schedule indicating events occurring in certain spaces of the school building at certain times with certain students of the plurality of students; and
implementing, by the processing circuit, responsive to determining the space infection level of the space is above a predefined amount, at least one of scheduling cleaning for the space, implementing updated environmental control of the space, or recommending additional filtration equipment for the space.

27. The method of claim 23, further comprising:
determining, by the processing circuit, based on the attendance data, the infection risk level of a plurality of groups of students of the school building; and
recommending, by the processing circuit, one or more first groups of students of the plurality of groups of students attend in person class and one or more second groups of students of the plurality of groups of students attend remote class based on the infection risk level of the plurality of groups of students.

28. The method of claim 23, wherein the one or more operations include operating one or more pieces of equipment in the school building to reduce the infection risk level.

29. The method of claim 23, wherein the one or more operations include determining an infectious disease prevention policy recommendation for the school building using the infection risk level.

30. A building system for a building, the building system including one or more memory devices storing instructions thereon that, when executed by the one or more processors, cause the one or more processors to:
receive attendance data indicating whether a plurality of occupants of the building are present or absent from the building;
determine, based on the attendance data, an infection risk level of at least one of the building, a space of the building, or one or more of the plurality of occupants to being infected with an infectious disease present in a population; and
perform one or more operations for causing the infection risk level to be reduced in the building.
